(19) United States
(12) Patent Application Publication
(10) Pub. No.: US 2006/0210476 A1
(43) Pub. Date: Sep. 21, 2006

(54) Metabonomics homogeneity analysis

(76) Inventors: Glenn H. Cantor, Princeton, NJ (US); Vikram Roongta, Belle Mead, NJ (US); Nelly Aranibar, Lawrenceville, NJ (US); Steven J. Bulera, Camillus, NY (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 11/368,978

(22) Filed: Mar. 6, 2006

Related U.S. Application Data
(60) Provisional application No. 60/662,120, filed on Mar. 15, 2005.

(51) Int. Cl. A61K 49/00; G06F 19/00; A61B 5/05

(52) U.S. Cl. 424/9.2; 702/19; 600/412

(57) ABSTRACT

A metabonomic method of selecting one or more non-human primate subjects for inclusion in a study is disclosed. The method generally involves spectroscopically profiling a sample of bodily fluid acquired from a subject proposed to be included in the study. The subject is accepted or rejected as a member of the proposed study based on a chemometric analysis of the similarities and differences between the subjects' samples, which provides a homogeneous subject pool for the study. The method can be applied to any type of subject, for example, non-human primates.

This application claims benefit to provisional application U.S. Ser. No. 60/662,120 filed Mar. 15, 2005, under 35 U.S.C. 119(e). The entire teachings of the referenced application is incorporated herein by reference.

### FIELD OF THE INVENTION

The present invention relates generally to metabonomic methods. More particularly, the invention relates to a method of determining the homogeneity of a population of candidates for a study, such as a preclinical or non-clinical study. The invention also relates to methods of analyzing and optimizing the homogeneity of a population of proposed subjects.

### BACKGROUND

Metabonomics is, generally, the study of the patterns of expression of endogenous metabolites in the body. Typically, this is done on body fluids such as serum, plasma, urine, or other fluids, although it is also possible to do metabonomics analyses on solid tissues. Typically, endogenous metabolites are examined by proton-nuclear magnetic resonance (“¹H-NMR”), liquid chromatography-mass spectroscopy (“LC-MS”), and other analytic chemical techniques. These techniques enable simultaneous detection of numerous endogenous metabolites in a non-biased way. The patterns of peaks revealed by these analytical techniques can then be summarized by multivariate analysis so that each animal's endogenous metabolites can be quantitatively compared with those of other animals.

Non-human primates and other animals are commonly used in preclinical and non-clinical toxicology studies to predict drug safety liabilities in human patients. In the case of monkeys, these animals are usually either caught in the wild or raised under semi-wild conditions, and are generally heterogeneous, due to genetic differences, underlying subclinical diseases, and other individual variations. Typically, because of the expense associated with monkeys, as well as their availability, the numbers used in studies are smaller than the numbers of rodents used in similar studies. The combined effect of a smaller test population and the variation among monkeys can statistically skew the results of preclinical and/or non-clinical studies and cause researchers to discard otherwise useful drug candidates.

In laboratory rodents such as mice and rats, there is much less genetic variation. Specific strains of rodents are purpose-bred under carefully controlled laboratory conditions and seldom exposed to disease pathogens. As compared with non-human primates, large numbers of mice and rats are available for research at low cost. Accordingly, it is common for laboratory studies to use larger numbers of rodents and the impact of each individual on the overall study conclusions is lower. Often, however, rodent models are not satisfactorily representative of humans and their use can, therefore, be of limited value.

In preclinical and non-clinical studies, it is preferable to optimize study outcomes by selecting non-human primates that are as homogeneous as possible. Often, non-human primates are screened before studies commence, using, for example, behavioral observations, physical examinations, and a profile of clinical pathology parameters. These conventional methods can detect some causes of heterogeneity among subjects in a test population, but it would be desirable to detect additional factors that can confound studies, including biochemical and metabolic differences among subjects.

What is needed, therefore, is a more comprehensive test to better determine the homogeneity among a population of laboratory subjects, such as non-human primates, before subjecting the animals to laboratory testing and research. An improved test would allow the investigator to better distinguish between acceptable and unacceptable subjects for a study. The present invention addresses this and other problems.

### SUMMARY OF THE INVENTION

A metabonomic method of selecting one or more non-human primate subjects for inclusion in a study from a population of proposed subjects is disclosed. In one embodiment, the method comprises: (a) acquiring a sample comprising a bodily fluid from a proposed subject; (b) generating a component profile spectrum of the sample; (c) analyzing the component profile spectrum of the sample using a chemometric technique to identify one or more spectral features selected from the group consisting of: (i) the presence of one or more spectral peaks characteristic of one or more chemical components of the sample; (ii) the absence of one or more spectral peaks characteristic of one or more chemical components of the sample; (iii) the relative distribution of one or more spectral peaks characteristic of one or more chemical components of the sample; (iv) the intensity of one or more spectral peaks characteristic of one or more chemical components of the sample; and (v) the position of one or more spectral peaks characteristic of one or more chemical components of the sample; (d) repeating steps (a) through (c) for each proposed subject; and (e) selecting for inclusion in the study those subjects from whom the acquired samples exhibit similar spectral features.

In the method, the non-human primates can be any non-human primates, including cynomolgus monkeys. Further, any body fluid samples can be employed in the method for example blood serum, blood plasma, or urine.

The component profile spectrum can be generated by employing any suitable analytic technique, such as a technique selected from the group consisting of ¹H-NMR, ¹³C-NMR, ¹⁵N-NMR, ³¹P-NMR, liquid chromatography, mass spectroscopy, gas chromatography and combinations thereof. When ¹H-NMR is selected as the analytical technique, the technique can comprise employing a pulse sequence that reduces a spectral contribution arising from one or more large molecular weight components, such as proteins and lipoproteins. Examples of such a pulse sequence include a Carr-Purcell-Meiboom-Gill (CPMG) pulse sequence and a pulse sequence comprising excitation sculpting pulse sequences preceded by an adiabatic presaturation pulse. A pulse sequence that reduces spectral contributions arising from water can also be employed

In the method, a chemometric technique is employed. The chemometric technique can be selected from the group consisting of a supervised multivariate method and a principal component analysis (PCA), for example. When a supervised multivariate method is employed, the method can be a partial-least-squares discriminant analysis. The analyzing can be performed on a selected region of the component profile spectrum or it can encompass the full range of the spectrum.

Thus, it is an object of the present invention to provide a metabonomic method of selecting one or more non-human primate subjects for inclusion in a study from a population of proposed subjects.

An object of the invention having been stated hereinabove, other objects will be evident as the description proceeds, when taken in connection with the accompanying Drawings and Examples as described herein.

### BRIEF DESCRIPTION OF THE DRAWINGS

FIG. 1 depicts two NMR spectra of a serum sample acquired from a monkey; the upper spectrum was generated using a sculpted pulse sequence and the lower spectrum was generated using the CPMG pulse sequence.

FIG. 2 is an NMR spectrum and highlights NMR assignments that were made based on chemical shift and multiplicity in the upfield, aliphatic region of the NMR spectrum of FIG. 1.

FIG. 3 is an NMR spectrum and highlights NMR assignments that were made based on chemical shift and multiplicity in the sugar region, and highlights the alpha proton resonances of amino acids, in addition to other resonances observed in the serum NMR spectrum of FIG. 1.

FIG. 4 is an NMR spectrum and highlights NMR assignments that were made based on chemical shift and multiplicity in the aromatic region of the serum NMR spectrum of FIG. 1.

FIG. 5 is a plot depicting a PCA mapping for the first three principal components of all monkey serum spectra recorded with the CPMG pulse sequence (small molecules); triangles represent females and squares represent males.

FIG. 6 is a plot depicting a PCA mapping for the first three principal components of all monkey serum spectra recorded with the excitation sculpting method, which captures the small and large (e.g., protein and lipoproteins) resonances.

FIG. 7 is a plot depicting a partial least square discriminant analysis (PLS-DA) based on gender.

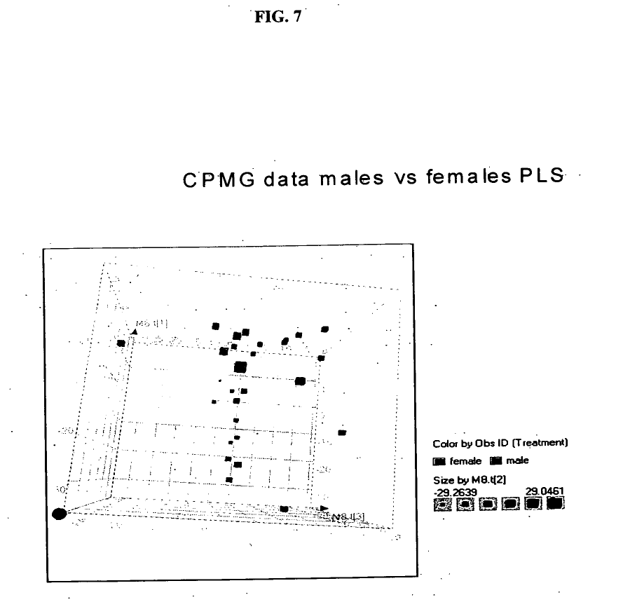

FIG. 8 is a plot depicting the variability, expressed as the standard deviation as a function of NMR chemical shift value, plotted together with the mean for both gender groups.

FIG. 9 is a plot depicting the observation that samples clustered together, indicating homogeneity; a principal component analysis (PCA) was employed in the analysis and principal components (PC) 1, 2, and 3 are shown in the figure; a sculpted NMR pulse sequence was employed in the generation of the spectral data.

FIG. 10 is a plot depicting the observation that samples clustered together, indicating homogeneity; a principal component analysis (PCA) was employed in the analysis and principal components (PC) 1, 2, and 3 are shown in the figure; a CPMG NMR pulse sequence was employed.

### DETAILED DESCRIPTION OF THE INVENTION

In one aspect, the present invention relates to metabonomic methods for assessing and/or ensuring the homogeneity of a population of subjects. Homogeneous populations of subjects are particularly desirable when preparing a preclinical or non-clinical trial. The method is particularly useful for assessing and/or ensuring the homogeneity of a population of laboratory animals, notably non-human primates. Ensuring the homogeneity of a population of non-human primates employed in a preclinical or non-clinical trial can reduce the overall costs of the trial, in addition to reducing the number of animals that are required for the trial.

### I. Definitions

Following long-standing patent law convention, the terms “a” and “an” mean “one or more” when used in this application, including the claims.

As used herein, the term “bodily fluid” means any fluid derived from a subject. A bodily fluid can be, but is not limited to, blood serum, plasma, whole blood, urine, and saliva.

As used herein, the term “metabonomics,” and grammatical derivations thereof, is used interchangeably with the terms “metabolic profiling” and “metabolomics” and describes the quantitative or qualitative multiparametric study of endogenous metabolite levels and/or patterns in an organism.

As used herein, the term “component profile spectrum” means a spectrum that is representative of one or more components of a sample. A component profile spectrum can be generated using any analytical technique, including, but not limited to, NMR, mass spectroscopy, absorption spectroscopy, gas chromatography, liquid chromatography, infrared spectroscopy, and combinations thereof.

As used herein, the term “spectral feature” means any distinctive aspect of a spectrum. A non-limiting list of examples of spectral features include peak height, peak width, peak area, peak position, peak presence, peak absence, and the ratios of features of a peak to the features of other peaks in the spectrum, as well as the ratios of one peak in the spectrum of a sample to the same peak in another sample.

### II. Representative Metabonomic Method of the Present Invention

The present invention generally relates to metabonomic applications, particularly in the context of their application to non-human primates. In one embodiment of the present invention, a metabonomic method of determining the relative homogeneity of a population of non-human primates is disclosed. The method can be employed in a pre-clinical or non-clinical application, for example, and can be used to identify subjects that may not be representative of the entire population of subjects. Traditionally, such an analysis is not performed until after pre-clinical or non-clinical data is acquired and analyzed and it is recognized that at least one subject included in the study should have been excluded. This can lead to increased costs, delayed results, and the need to repeat studies. This embodiment of the present invention will, therefore, find applications in pre-clinical and non-clinical scenarios, as well as in other situations in which it is desired to determine the relative homogeneity of a population of subjects, such as a toxicology study.

In one embodiment, the present invention discloses a metabonomic method of selecting one or more non-human primate subjects for inclusion in a study from a population of proposed subjects. In this method, a sample comprising a bodily fluid is first acquired from a proposed subject. The bodily fluid can be any fluid extracted from the proposed subject and can be, for example, blood serum, plasma, whole blood or urine. As noted herein, the method can be applied to any non-human primate, such as cynomolgus monkeys.

Although in some cases it may be desirable to purify a sample before performing a metabonomic method of the present invention, there is no requirement that the sample be purified prior to analysis. This is an advantage of the present invention and makes the present invention particularly amenable to a rapid assessment of a population of proposed subjects. In some cases, some sample preparation is desirable, such as when the bodily fluid examined is blood serum and it is necessary to remove red blood cells from a sample of whole blood.

Continuing with the method, after the sample has been acquired and any preparatory work has been performed, a component profile spectrum of the sample can be generated. A “component profile spectrum” is a spectrum that fully or partially reflects the chemical composition of one or more components in the sample. Preferably, but not necessarily, a component profile spectrum is a reflection of all of the discrete chemical components of the sample.

The precise nature of a component profile spectrum is variable, and will depend on the analytical technique employed in the method. For example, if NMR spectroscopy is employed to generate a component spectrum, then the component spectrum will be presented in terms of the nucleus for which the NMR probe is tuned; similarly, if mass spectroscopy is employed to generate a component spectrum, then the component spectrum will be presented in terms of the molecular weights of any species present in the sample, and fragments thereof.

Any suitable technique can be used to generate a component profile spectrum of the sample. Representative, but non-limiting, examples of techniques that can be employed in the methods of the present invention include NMR spectroscopy, gas chromatography, liquid chromatography, mass spectrometry and combinations thereof. These analytical techniques are well-known to those of ordinary skill in the art. Variations and modifications to these basic methods can be made and employed in the present invention; such variations will be a function of the technique selected.

Although any analytical technique can be employed in the present invention to generate a component spectrum, component spectrum generation is particularly suited to NMR spectroscopy. One means of acquiring an NMR spectrum involves the application of a strong radio frequency (RF) pulse of energy over the whole range of frequencies while the magnetic field is kept constant. As a result, nuclei are flipped to their higher energy state from which, over time, they will return (decay) to the lower state and generate an induced current. Acquiring the induced current as a function of time through a computer creates a time-domain signal, which is a generally complex pattern called a free-induction decay (FID). A Fourier transformation of an FID yields an interpretable spectrum.

When NMR is employed to generate a component profile spectrum, any suitable pulse sequence can be employed, and the selection of a pulse sequence can be, in part, a function of the nature of the sample. For example, in the case of aqueous samples it may be desirable to employ a water suppression pulse sequence in addition to an acquisition pulse sequence. One example of a pulse sequence that can be employed in the present invention is the Carr-Purcell-Meiboom-Gill (CPMG) pulse sequence, which is known in the art (Carr & Purcell, (1954) Phys. Rev. 94:630-38; Meiboom & Gill, (1958) Rev. Sci. Instrum. 29:688-91). Pulse programs for the CPMG sequence and other pulse sequences are available online or as part of a commercially-available software package. Additionally, there is no limit on the nucleus selected for the acquisition of a component profile spectrum; most often it will be desirable to acquire ¹³H or ³C spectra in the context of the present invention, due to the relatively strong signal strength associated with the nuclei, but ¹⁵N, ³¹P or other biologically significant nucleus can also be selected for the a component profile spectrum.

Continuing with the method, after a component profile spectrum of the sample has been generated, the component spectrum is analyzed using a chemometric technique to identify one or more spectral features selected from the group consisting of: (i) the presence of one or more spectral peaks characteristic of one or more chemical components of the sample; (ii) the absence of one or more spectral peaks characteristic of one or more chemical components of the sample; (iii) the relative distribution of one or more spectral peaks characteristic of one or more chemical components of the sample; (iv) the intensity of one or more spectral peaks characteristic of one or more chemical components of the sample; and (v) the position of one or more spectral peaks characteristic of one or more chemical components of the sample.

Chemometrics is a chemical discipline that employs mathematical and statistical methods to relate measurements made on a chemical system to the state of the system and to design or select optimal measurement procedures and experiments. Stated another way, the field of chemometrics is the application of statistical and mathematical techniques to the analysis of chemical data.

Any chemometric technique can be employed in the present invention. Examples of chemometric techniques that can be employed in the present invention include, but are not limited to, Principal Component Analysis (PCA), Partial Least Squares Regression (PLS), Principal Components Regression (PCR), Multilinear Regression Analysis (MLR) and Discriminant Analysis.

In a PCA operation, a set of correlated variables is compressed into a smaller set of uncorrelated variables. This transformation consists of a rotation of the coordinate system, resulting in the alignment of information on a fewer number of axes than in the original arrangement. In this way, variables that are highly correlated with one another are treated as a single entity. By using PCA, it is possible to obtain a small set of uncorrelated variables still representing most of the information which was present in the original set of variables, but in a form that is easier to employ.

PLS is a modeling and computational method by which quantitative relations can be established between groups of variables. One advantage of PLS is that the results can be evaluated graphically, by different plots. In many cases, visual interpretations of the plot are sufficient to obtain a good understanding of different relations between the variables.

PCR is related to PCA and PLS. As in PCA, each object in one group is projected onto a lower dimensional space yielding scores and loadings. The scores are then regressed against the response block in a least squares procedure leading to a regression model which can be used to predict unknown samples. The same model statistics employed in PLS and PCA can be used to validate the model.

In a MLR analysis, the best fitting plane for the parameters as a function of the spectra is defined, using least squares techniques to define each boundary of the plane. This plane is then used to recognize and assign a predicted value to an unknown parameter value. This is a method whereby, by use of spectral data, the known parameter values are grouped into different clusters, separated by linear decision boundaries. In terms of a spectrum, a sample of unknown parameter values then can be matched to a cluster, and the parameter value can be assigned a value, e.g. the average value of the cluster. This is a very useful technique for quality screening.

Any of the above chemometric techniques can be employed in a method of the present invention. The chemometric technique can be employed to identify one or more spectral features of the component spectrum, including the presence of one or more spectral peaks characteristic of one or more chemical components of the sample. The one or more peaks can comprise, for example, a distinct and identifiable peak(s) in an NMR spectrum that is known or suspected to be attributable to a chemical component of a sample. For example, if it is desired to detect the presence of a particular molecule (e.g., an amino acid, a particular organic anion or a metabolite) a chemometric technique can be employed to identify one or more spectral peaks, e.g., peaks in an NMR spectrum or in a mass spectroscopy spectrum, that are characteristic of the molecule. In a related example, such peaks can also be indicative of a chemical species that is a metabolic byproduct of the molecule, e.g., indicative of a change in the equilibrium between biosynthesis and catabolism of that particular metabolite in the organism.

In another example, a chemometric technique can be employed to identify the absence of one or more spectral peaks characteristic of one or more chemical components of the sample. Again, the one or more peaks can comprise, for example, a distinct and identifiable peak(s) in an NMR spectrum that is known or suspected to be attributable to a chemical component of a sample. For example, if it is desired to detect the absence of a molecule (e.g., an amino acid, a particular organic anion or a metabolite), a chemometric technique can be employed to identify the spectral position(s) at which the peak(s) would be expected to appear, e.g., peaks in an NMR spectrum or in a mass spectroscopy spectrum, that are characteristic of the molecule. In a related example, such peaks can also be indicative of the absence of the biosynthesis of the molecule.

In a further example, a chemometric technique can be employed to identify the relative distribution of one or more spectral peaks characteristic of one or more chemical components of the sample. The one or more peaks can comprise, for example, a distinct and identifiable peak(s) in an NMR spectrum that is known or suspected to be attributable to a chemical component of a sample. For example, if it is desired to detect the presence of a particular molecule (e.g., an amino acid, a particular organic anion or a metabolite), a chemometric technique can be employed to identify one or more spectral peaks, e.g., peaks in an NMR spectrum or in a mass spectroscopy spectrum, that are characteristic of the molecule. In a related example, such spectral peaks can also be indicative of a chemical species that is a component in the same or a related metabolic pathway. After identifying the peaks, the distribution of these peaks relative to one another can be determined. The relative distribution encompasses the presence of the peaks, as well as other qualitative or quantitative traits that can be compared. Stated another way, the chemometric technique can be employed to determine the presence or absence of one or more characteristic peaks as well as other properties of the peaks, which are gauged relative to one another.

In yet another example, a chemometric technique can be employed to identify the intensity of one or more spectral peaks, e.g., peaks in an NMR spectrum or in a mass spectroscopy spectrum, that is characteristic of one or more chemical components of the sample. Again, this application of the chemometric technique can be employed to identify not only the presence of one or more spectral peaks that are characteristic of a particular molecule (e.g., an amino acid or a particular organic anion), but also the intensities of these characteristic peaks, relative to each other and/or to one or more other peaks in the component spectrum.

In a further example, a chemometric technique can be employed to identify the position of one or more spectral peaks, e.g., peaks in an NMR spectrum or in a mass spectroscopy spectrum, that are characteristic of one or more chemical components of the sample. In this comparison, the positions of the spectral peaks in the component spectrum relative to one or more spectral peaks present in the spectrum is the basis of the comparison. The peaks against which the positions of the spectral peaks are gauged can be derived from a single chemical species or from two or more chemical species.

Continuing, the above-described steps can be repeated for each proposed subject. The number of proposed subjects can vary and can be of any number, although proposed subject populations of comprising larger numbers of subjects are generally preferable and are more amenable to significant statistical analyses. It is noted, however, that any size of three or more subjects in the proposed subject population can be studied using the present invention, with the understanding that smaller populations may present complications for statistical analyses. After each repetition of the steps, the results of the chemometric analysis can be stored for subsequent analysis and used as the basis for including or excluding a proposed subject from the study. These data can be stored in any convenient form, such as electronically, e.g., on computer discs or a hard drive in a database, or on paper as hardcopies.

After the above-described steps have been performed for each member of the population, those subjects from whom the acquired samples exhibit similar spectral features are selected for inclusion in the study. The results of the chemometric analysis performed on each subject can be analyzed in order to identify those samples exhibiting similar spectral features; those subjects from whom samples exhibiting similar spectral features were obtained can be included in the study while those subjects from whom samples exhibiting non-similar spectral features were obtained can be excluded from the study.

Generally, the analysis employed in the identification of samples having similar spectral features can be performed by comparing the presence, absence or relative positions of the spectral features within the dataset of analyzed component profile spectra to each other. The comparison can be made using any means and can be automated, such as a computer-based analysis, or it can simply be by visual inspection.

The application of the methods of the present invention can facilitate the generation of a homogeneous subject pool. This is achieved because outliers, i.e., subjects whose samples are not homogeneous with the other members of the population, can be removed from the study at an early stage. The early exclusion of outliers can save time and money by removing the necessity to repeat a study due to the inadvertent inclusion of an outlier, can conserve resources, including economic resources and biological resources, and can increase the overall efficiency of a study. For example, excluding outliers has the effect of removing variability from a study and increasing the confidence level and significance of animal study findings.

### EXAMPLES

The following Examples have been included to illustrate various exemplary modes of the invention. Certain aspects of the following Examples are described in terms of techniques and procedures found or contemplated by the inventors to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications and alterations can be employed without departing from the spirit and scope of the invention.

### Example 1

Monkeys, unlike rodents, have a more varied genetic background and, with the high cost and the limited number of monkeys that can be used for drug safety testing, it is advantageous to have a simple, fast screen in place to avoid sick or abnormal animals from being placed on an in vivo study. The present example demonstrates that serum metabonomics by NMR is a viable screen to detect abnormal states in the animals prior to a study.

Serum samples from naive and non-naive, male and female cynomolgus monkeys were measured by NMR and the spectra were analyzed by multivariate non-supervised and supervised statistical methods. No major differences were found between the non-naive and naive animals, and the variability between males and females was comparable. One animal seemed to be an outlier, as determined by PCA. A weak distinction between males and females could be forced with supervised multivariate methods (PLS-DA) but the separation did not cross-validate well. It was possible to assign a majority of the dominating resonances to common metabolites.

The spectra were of high quality and highly consistent. The NMR spectrum of a serum sample quantitatively measures the concentration of a multitude of metabolites contained in the serum. In addition, using alternative NMR pulse sequences, the protein resonance signals could be measured, which may also contain some profile information. It has been exemplified in the literature that NMR spectral profiles of serum samples are related with certain disease states, like cancer. Also, lipid profile changes are routinely quantified by NMR. Therefore, this method can be a valuable and cost effective tool for prescreening monkeys and other laboratory animals.

### Methods for Example 1

Serum samples from 16 males and 16 female monkeys were submitted for analysis by Drug Safety Evaluation, Syracuse, N.Y. From a total of 32 samples, 10 females were non-naive and had received various treatments. The last treatment was, however, more than 4 weeks before the bleed for metabonomics. The animals had been anesthetized with ketamine (10-20 mg/kg) for the blood collection.

NMR proton spectra of each group were recorded on a 600 MHz Bruker spectrometer. Different NMR pulse sequences were used for these samples. The CPMG (Carr-Purcell-Meiboom-Gill) pulse sequence was used to filter out the resonances of the large molecular weight components in the plasma (e.g., protein, and lipoprotein peaks). Otherwise, excitation sculpting sequences were used so as to have the total range of resonances, low and high molecular weight. The excitation sculpting provided optimal water resonance suppression. For the NMR measurements, 0.4 ml of serum was diluted with a 0.2 ml of a 2M PBS buffer in D₂O to obtain a final volume of 0.6 ml, supplemented with 1 mM TSP and 0.1% w/v sodium azide. A total of 256 scans were accumulated for the excitation sculpting sequences and 512 for the CPMG filtered data to compensate for the lower sensitivity of this method. Total spectrometer time, including equilibration, for collection of one spectrum was 15 minutes. Unsupervised principal component analysis (PCA) and supervised partial least square-discriminant analysis (PLS-DA) were applied to the spectra after binning into 1K points, and zeroing the water residual peak scaling to constant total integral, and normalization by mean centering and univariant scaling. A two-way Analysis of Variance (ANOVA) was also performed on the scaled, but not normalized, data.

### Results from Example 1

The processing of the samples closely followed typical protocols for handling serum samples, and care was taken to avoid variations in sample handling. For the acquisition of the NMR data, we used the CPMG pulse sequence as a filter to remove broad resonances such as those from proteins or lipids. A novel water suppression technique was employed that was pioneered by the present inventors (N. Aranibar, K.-H. Ott, L. Mueller, N. Contel, V. Roongta, (2003) “Comparison of Water Suppression Techniques for Metabonomics” (abstract), 44th Experimental NMR Conference.) specifically for metabonomics and which has been applied to a variety of other biofluids.

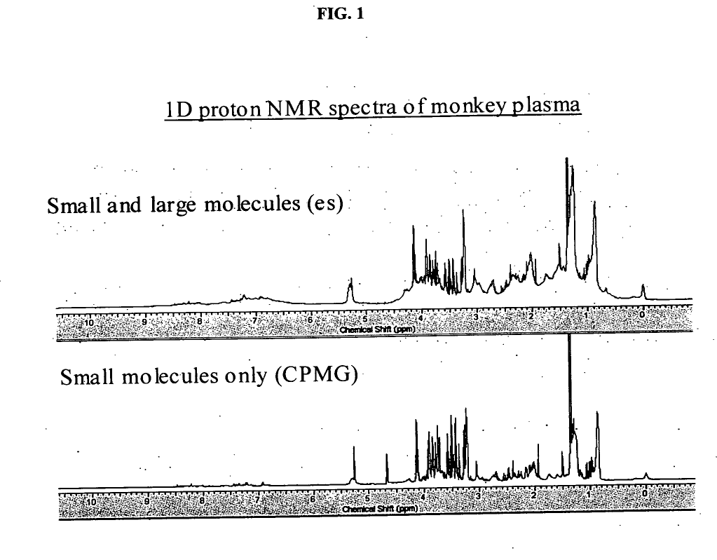

FIG. 1 shows a comparison of a serum NMR spectrum acquired with the two different pulse sequences. The NMR spectra of serum using the T2 filtering sequence allows the assignment of many resonances. The upper spectrum in FIG. 1 demonstrates the observation that excitation sculpting effectively removes the large water resonance without disturbing the remainder of the spectrum. The lower spectrum of FIG. 1 demonstrates the observation that filtering relaxation-broadened resonances by the CPMG pulse train removes the large humps originating from the proton resonances of large biomolecules. A reduction in the number of lipid resonances (˜0.8 ppm and 1.2 ppm), most oligosaccharides (˜3.5 ppm) and the almost complete disappearance of resonances of protein amino acids (1 ppm-5 ppm and 6 ppm-9 ppm.) is depicted.

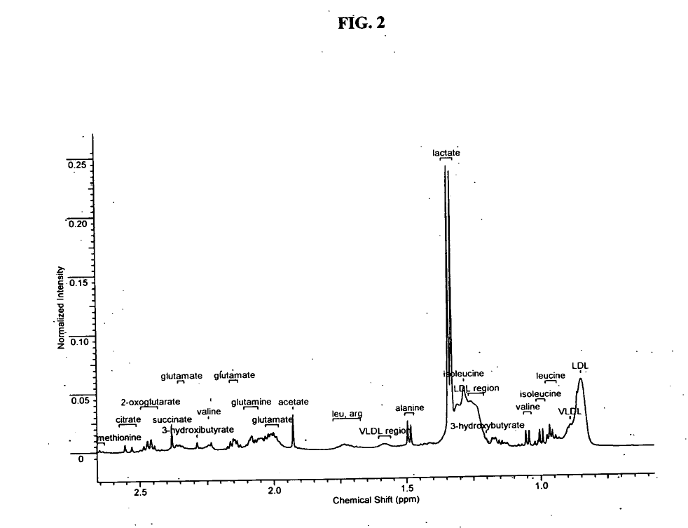

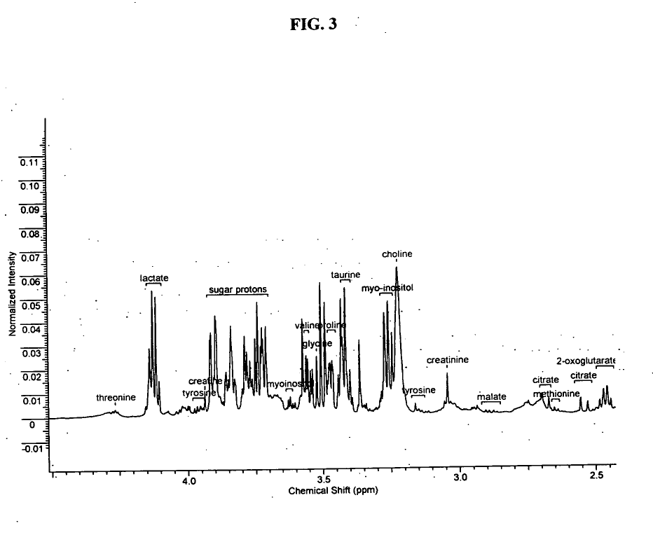

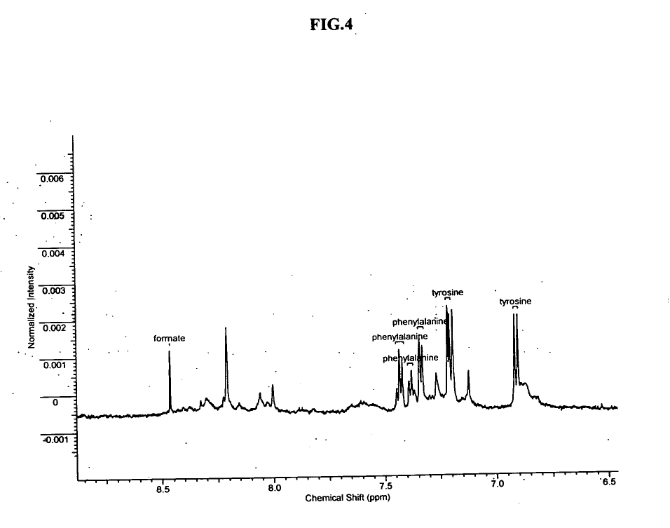

The tentative assignments of chemical shift values that are indicated in FIGS. 2 through 4 are based on values from the literature and spectra of standard compounds in water. Definitive assignments can be accomplished by spiking standard compounds into the serum sample and/or 2D NMR techniques. There were no obvious signals found for the anesthetic administered to the monkeys, namely ketamine.

### Conclusions from Example 1

CPMG and excitation sculpting NMR techniques provide complementary information. CPMG removes the envelope of large biomolecules thus reducing the complexity of the spectrum but is less sensitive due intensity loss by relaxation inherent to the pulse sequence. The sensitivity reduction can be compensated for by increasing the number of scans accumulated during acquisition.

It was observed that about 50 different common metabolites can be readily identified by reference to our compendium of chemical shift values for reference compounds. Spiking and 2D TOCSY NMR spectroscopy can facilitate the unique identification of these compounds and others.

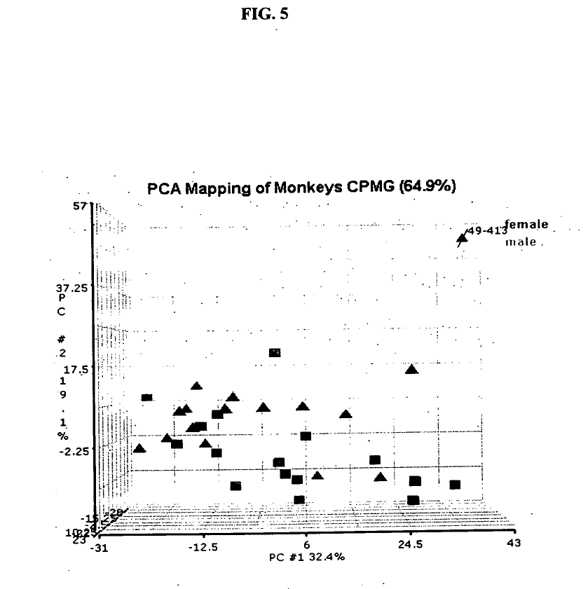

It was observed that NMR spectra of male and female monkey plasma samples do not differ significantly (see FIG. 5).

In this study set, monkey serum from animals that have been treated previously but that had a recovery time of at least four weeks did not differ from the naive group.

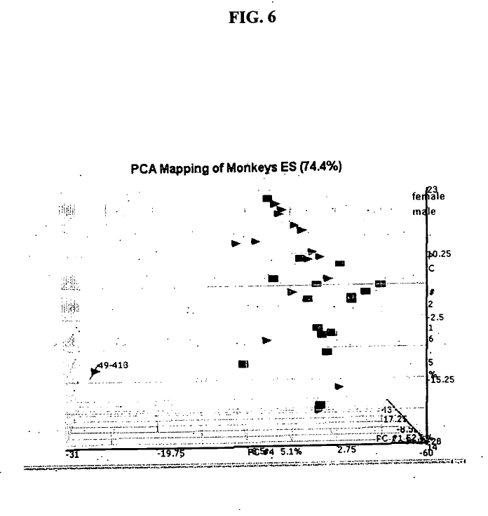

A single outlier could be identified from serum spectra in this study based on PCA scores (FIG. 5). Note that this is not a homogeneous population; subject 49-413 maps differently from the other subjects. Additional measurements on the outlier animal showed a broadening of a series of lipid and lipoprotein related resonances. FIG. 6, a PCA plot, shows that there is no obvious grouping of the samples by gender, although sample 49-413 appears to be an outlier in PCA space; as in FIG. 5, subject 49-413 maps differently from the other subjects.

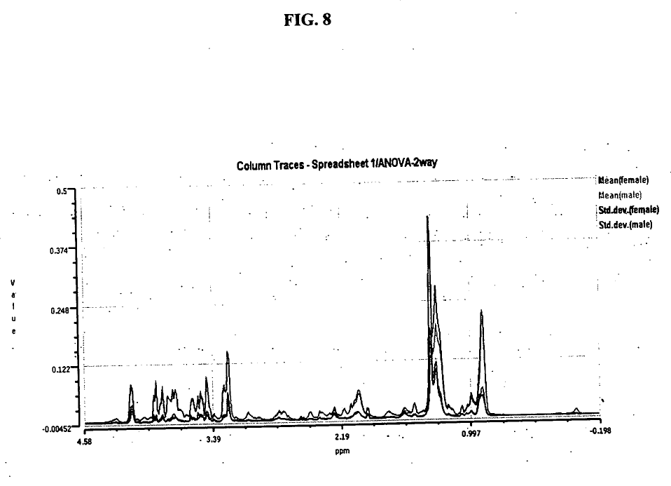

FIG. 8 demonstrates the variability between the serum spectra of males and females is comparable. Different resonances have a variable relative magnitude of standard deviation. For example, lipids (˜1.2 ppm) vary strongly (˜standard deviation is ˜50% of mean intensity), while other components are tightly regulated.

### Example 2

A goal of this Example was to select a relatively homogeneous population of cynomolgus monkeys for toxicology studies. This was achieved by comparing the patterns of endogenous metabolites in serum samples.

Serum samples from 15 male and 15 female cynomolgus monkeys were examined by ¹H-NMR and the spectra were analyzed by principal component analysis (PCA). Three major components in the PCA were displayed graphically and visually examined for homogeneity. Two types of pulse sequences were used: (1) excitation sculpting water suppression (WGL, preceded by an adiabatic presaturation) to show both low- and high-molecular-weight molecules including proteins and lipoproteins, and (2) Carr-Purcell-Meiboom-Gill (CPMG) to focus on the small molecular-weight molecules.

The 30 serum samples formed a homogeneous population based on PCA, and no major differences were found among animals. Based on these results, all of the animals are suitable for inclusion in the studies.

### Results from Example 2

Serum samples from 15 males and 15 female cynomolgus monkeys were submitted for analysis. The animals are listed in Table 1. Blood samples were collected and measured and analyzed within five days or less of collection.

TABLE 1

| Tattoo # | Alternate ID | Sex | DOB | Sample comments |
|---|---|---|---|---|
| 121-859 | 3169 | F | Jul-00 | |
| 13-004 | 3155 | M | Aug-01 | |
| 13-221 | 3128 | F | Aug-01 | hemolyzed |
| 13-253 | 3129 | F | Jul-01 | hemolyzed |
| 13-304 | 3130 | F | Jul-01 | |
| 13-338 | 3131 | F | May-01 | |
| 42-188 | 3154 | M | Dec-00 | |
| 43-067 | 3173 | F | Dec-01 | |
| 43-073 | 3181 | F | Nov-01 | |
| 43-074 | 3177 | F | Dec-01 | |
| 43-101 | 3161 | M | Dec-01 | |
| 63-021 | 3164 | M | Nov-01 | |
| 63-022 | 3163 | M | Dec-01 | hemolyzed |
| 63-023 | 3183 | F | Dec-01 | |
| 63-029 | 3185 | F | Dec-01 | |
| 63-032 | 3132 | F | Nov-01 | |
| 63-035 | 3133 | F | Dec-01 | |
| 63-042 | 3189 | F | Jan-02 | |
| 93-016 | 3126 | F | May-02 | |
| 93-019 | 3127 | F | Jul-02 | |
| 93-029 | 3092 | M | May-02 | |
| 93-031 | 3145 | M | Jun-02 | |
| 93-032 | 3146 | M | May-02 | |
| 93-033 | 3093 | M | Mar-02 | |
| 93-034 | 3165 | M | May-02 | |
| 93-036 | 3095 | M | May-02 | |
| 93-038 | 3147 | M | May-02 | |
| 93-039 | 3148 | M | May-02 | |
| 93-040 | 3149 | M | May-02 | |
| 93-041 | 3150 | M | May-02 | |

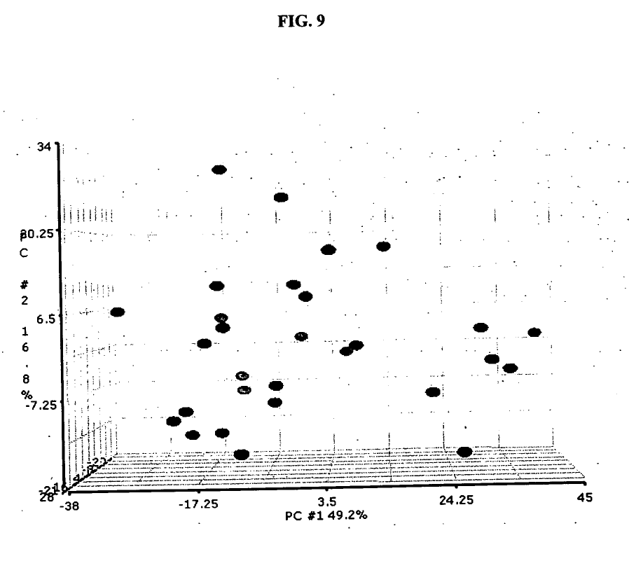

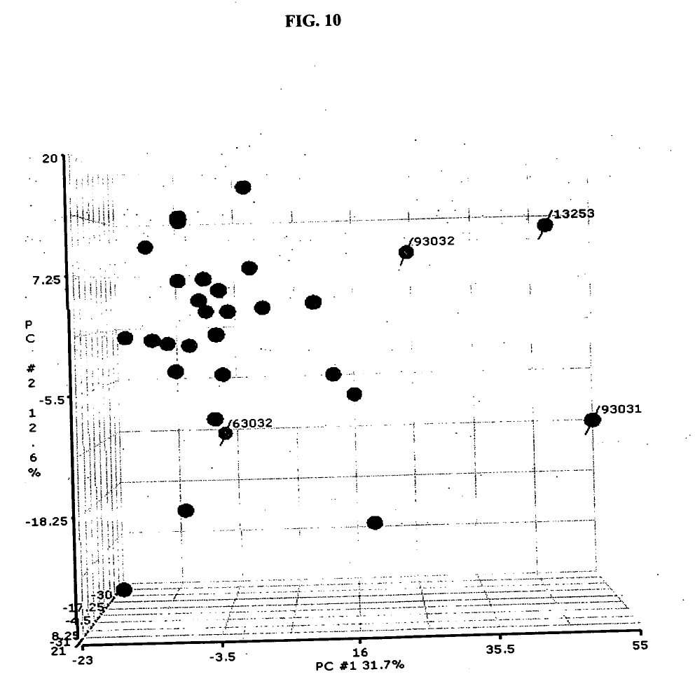

The 30 serum samples formed a homogeneous population based on visual inspection of PCA, and no major differences were found among animals (FIGS. 9 and 10). FIG. 9 indicates that the use of the excitation sculpting water-suppression (WGL) pulse sequence to show small and large molecular weight molecules, including proteins and lipoproteins, was effective. FIG. 10 indicates that the use of the CPMG pulse sequence to show small and large molecular weight molecules, including proteins and lipoproteins, was effective.

Three of the 30 serum samples were dark pink, indicating hemolysis. These samples were from animals 13-221 (female), 63-022 (male), and 13-253 (female). Despite hemolysis, these three samples grouped among the others.

### Conclusions from Example 2

The purpose of this metabonomics study is to select a relatively homogeneous population of cynomolgus monkeys for toxicology studies. This is done by comparing the patterns of endogenous metabolites in serum samples.

The 30 serum samples formed a homogeneous population based on PCA, and no major differences were found among animals. Based on these results, all of the animals are suitable for inclusion in the studies.

### REFERENCES

The references cited in the specification are incorporated herein by reference to the extent that they supplement, explain, provide a background for or teach methodology, techniques and/or compositions employed herein. All cited patents, including patent applications, and publications referred to in this application are herein expressly incorporated by reference.

It will be understood that various details of the invention may be changed without departing from the scope of the invention. Furthermore, the foregoing description is for the purpose of illustration only.

What is claimed is:

1. A metabonomic method of selecting one or more non-human primate subjects for inclusion in a study from a population of proposed subjects comprising:
(a) acquiring a sample comprising a bodily fluid from a proposed subject;
(b) generating a component profile spectrum of the sample;
(c) analyzing the component profile spectrum of the sample using a chemometric technique to identify one or more spectral features selected from the group consisting of:
(i) the presence of one or more spectral peaks characteristic of one or more chemical components of the sample;
(ii) the absence of one or more spectral peaks characteristic of one or more chemical components of the sample;
(iii) the relative distribution of one or more spectral peaks characteristic of one or more chemical components of the sample;
(iv) the intensity of one or more spectral peaks characteristic of one or more chemical components of the sample; and
(v) the position of one or more spectral peaks characteristic of one or more chemical components of the sample;
(d) repeating steps (a) through (c) for each proposed subject; and
(e) selecting for inclusion in the study those subjects from whom the acquired samples exhibit similar spectral features.

2. The method of claim 1, wherein the non-human primates are cynomolgus monkeys.

3. The method of claim 1, wherein the body fluid samples is selected from the group consisting of blood serum, blood plasma, and urine.

4. The method of claim 1, wherein the component profile spectrum is generated by employing a technique selected from the group consisting of ¹H-NMR, ¹³C-NMR, ¹⁵N-NMR, ³¹P-NMR, liquid chromatography, mass spectroscopy, gas chromatography and combinations thereof.

5. The method of claim 4, wherein the ¹H-NMR technique comprises employing a pulse sequence that reduces a spectral contribution arising from one or more large molecular weight components.

6. The method of claim 5, wherein the one or more large molecular weight components are selected from the group consisting of proteins and lipoproteins.

7. The method of claim 6, wherein the pulse sequence is selected from the group consisting of a Carr-Purcell-Meiboom-Gill (CPMG) pulse sequence and a pulse sequence comprising excitation sculpting pulse sequences preceded by an adiabatic presaturation.

8. The method of claim 4, wherein the ¹H-NMR technique comprises employing a pulse sequence that reduces spectral contributions arising from water.

9. The method of claim 1, wherein the chemometric technique is selected from the group consisting of a supervised multivariate method and a principal component analysis.

10. The method of claim 1, wherein the supervised multivariate method is a partial-least-squares discriminant analysis.

11. The method of claim 1, wherein the analyzing is performed on a selected region of the component profile spectrum.

12. The method of claim 1, wherein the study is selected from the group consisting of a preclinical study and a non-clinical study.
